(19) United States
(12) Patent Application Publication
(10) Pub. No.: US 2025/0134717 A1
(43) Pub. Date: May 1, 2025

(54) TRANSCLERAL LASER

(71) Applicants: National University of Singapore, Singapore (SG); National University Hospital (Singapore) PTE LTD, Singapore (SG)

(72) Inventors: Tec Kuan Paul CHEW, Singapore (SG); Teck Chang Victor KOH, Singapore (SG); Maria Cecilia Domingo AQUINO, Singapore (SG)

(21) Appl. No.: 18/927,116

(22) Filed: Oct. 25, 2024

Related U.S. Application Data
(60) Provisional application No. 63/593,480, filed on Oct. 26, 2023.

(51) Int. Cl. A61F 9/009; A61B 18/22; A61F 9/008

(52) U.S. Cl. CPC A61F 9/009; A61B 18/22; A61F 2009/00868

(57) ABSTRACT

Described are optical probes for application of laser light to at least the pars plana and optionally other regions, such as the trabecular meshwork, pars plicata, and/or ciliary sulcus, of an eye, such as for treatment of glaucoma, to reduce intraocular pressure (IOP), to improve outflow of aqueous humor from the eye, and/or reduce buildup of aqueous humor within the eye. The optical probes can be adapted for simultaneously treating multiple different regions, tissues, or bodies of the eye, or for sequentially treating multiple different regions, tissues, or bodies of the eye.

### CROSS-REFERENCES TO RELATED APPLICATIONS

This application claims the benefit and priority of U.S. Provisional Patent Application No. 63/593,480, filed Oct. 26, 2023, entitled “TRANSCLERAL LASER”, which is hereby incorporated by reference in its entirety.

### BACKGROUND OF THE INVENTION

Glaucoma is a common eye condition where the optic nerve becomes damaged. This is usually caused by fluid building up in the eye, which increases pressure inside the eye. Hence, to treat glaucoma, techniques can be used to reduce the pressure inside the eye, such as by creating a drainage pathway for the fluid in the eye. Various treatment regimes exist, but improved and alternative treatment methods are needed.

### BRIEF SUMMARY OF THE INVENTION

Described herein are optical probes useful for treating glaucoma and improving outflow of aqueous humor from the eye. The optical probes may be configured as a system including various components, such as a footplate, an optical waveguide coupled to the footplate, and a laser system coupled to waveguide. The optical probes may have a control system that carefully controls the power to ensure repeatable and consistent output of the laser light at a target level suitable for the desired treatment.

In one example, a system may comprise a footplate, the footplate shaped to mate with a surface of an eye, the footplate including one or more apertures; one or more optical waveguides coupled to the footplate and positioned to transmit laser light through the one or more apertures and expose at least a pars plana of the eye to laser light when the footplate is mated with the surface of the eye; and a laser system coupled to the one or more optical waveguides to emit laser light into the one or more optical waveguides for transmission to the eye through the one or more apertures.

Optionally, the footplate is shaped to mate with a sclera of the eye at or adjacent to a corneal limbus of the eye. In some examples, one or more dimensions of the footplate and/or a position of one or more apertures within the footplate are configured to position at least one aperture of the footplate above the pars plana when the footplate is mated to the surface of the eye. Such a configuration is not intended to be limiting and the footplate can have any suitable shape or dimensions that allow for treatment of the pars plana and other tissues, bodies, or regions of the eye, sequentially or simultaneously. In some examples, one or more dimensions of the footplate and/or a position of the one or more apertures within the footplate are configured to position at least one aperture of the footplate above the pars plana and at least one aperture of the footplate above a pars plicata of the eye when the footplate is mated to the surface of the eye. In some examples, one or more dimensions of the footplate and/or a position of the one or more apertures within the footplate are configured to position at least one aperture of the footplate above the pars plana and at least one aperture of the footplate above a trabecular meshwork of the eye when the footplate is mated to the surface of the eye. In some examples, one or more dimensions of the footplate and/or a position of the one or more apertures within the footplate are configured to position at least one aperture of the footplate above a pars plicata of the eye and at least one aperture of the footplate above a trabecular meshwork of the eye when the footplate is mated to the surface of the eye. In some examples, one or more dimensions of the footplate and/or a position of the one or more apertures within the footplate are configured to position a plurality of apertures of the footplate above the pars plana or above a pars plicata of the eye when the footplate is mated to the surface of the eye.

In some examples, the footplate is configured or adapted to be moved across a surface of the eye or to be repositioned at different positions on the eye for treating different tissues, bodies, or regions of the eye sequentially with laser light. Optionally, the footplate is adapted to move tangentially or circumferentially along the surface of the eye, parallel to the pars plana, to sequentially expose at least the pars plana in a plurality of quadrants of the eye to laser light. Optionally, the footplate is adapted to move tangentially or circumferentially along the surface of the eye, parallel to a ciliary sulcus of the eye, to sequentially expose at least the ciliary sulcus in a plurality of quadrants of the eye to laser light. Optionally, the footplate is adapted to move radially along the surface of the eye to expose the pars plana and a pars plicata of the eye to laser light. Optionally, radially along the surface of the eye to expose the pars plana, a pars plicata of the eye, and a trabecular meshwork of the eye to laser light. Without limitation, tangential or circumferential motion of the footplate may correspond to a translation along or above the surface of the eye about a pupil at a fixed radial position. Without limitation, radial motion of the footplate may correspond to a translation along or above the surface of the eye toward or away from the pupil at a fixed lateral position. In some examples, the footplate is adapted to move both radially and tangentially along or above the surface of the eye to expose the pars plana and a trabecular meshwork of the eye in a plurality of quadrants of the eye to laser light. Optionally, the footplate is adapted to move radially and tangentially along or above the surface of the eye to expose the pars plana, a pars plicata of the eye, and a trabecular meshwork of the eye to laser light. Optionally, the footplate is adapted to move both radially and tangentially in a zig-zag path along or above the surface of the eye.

As noted above, control over the output power of laser light emitted onto the eye can be achieved according to some examples of the systems described herein. For example, a system or optical probe may comprise a control system configured to control an output power of laser light emitted onto the eye. Example control systems include those configured to limit operation of the laser system to a fixed number of treatments to maintain a treatment power of the laser light above a threshold level.

Also described are methods, such as methods of operating an optical probe or system described herein. An example method comprises providing the optical probe or system; positioning the footplate of the optical probe or system against the surface of an eye; and causing actuation of the laser system to expose laser light onto at least the pars plana of the eye through the one or more apertures. Example methods may further moving the footplate of the optical probe from a first position against the surface of the eye to a second position against the surface of the eye; and causing actuation of the laser system to expose laser light onto the eye through the one or more apertures at the second position. In examples, exposing the laser light onto the eye through the one or more apertures at the second position includes exposing the pars plana, a trabecular meshwork of the eye, a pars plicata of the eye, and/or a ciliary sulcus of the eye to the laser light.

### BRIEF DESCRIPTION OF THE DRAWINGS

FIG. 1A provides a partial schematic cross-sectional illustration of an anterior portion of an eye, showing approximate positions of various tissues, bodies, and regions of the eye.

FIG. 1B provides a schematic front view illustration of an eye, showing the iris and pupil and approximate positions of various tissues, bodies, and regions of the eye with respect to the iris and pupil.

FIG. 2A provides a partial schematic cross-sectional illustration of an anterior portion of an eye, showing positioning of an example footplate against a surface of the eye for treating different tissues or regions of the eye with laser light.

FIG. 2B provides a schematic front illustration of an eye, showing the pupil and approximate positions of various tissues, bodies, and regions of the eye along with an example footplate positioned on a surface of the eye and showing circumferential motion of the footplate for treating different tissues or regions of the eye with laser light.

FIG. 2C provides a schematic illustration of an example footplate, showing a plurality of apertures and approximate positions within the footplate.

FIG. 3A provides a partial schematic cross-sectional illustration of an anterior portion of an eye, showing positioning of an example footplate against a surface of the eye for treating a tissue or region of the eye with laser light.

FIG. 3B provides a schematic front illustration of an eye, showing the pupil and approximate positions of various tissues, bodies, and regions of the eye along with an example footplate positioned on a surface of the eye and showing circumferential motion of the footplate for treating a full circumference of a single tissue or region of the eye with laser light.

FIG. 3C provides a schematic illustration of an example footplate, showing a plurality of apertures and approximate positions within the footplate.

FIG. 4A provides a partial schematic cross-sectional illustration of an anterior portion of an eye, showing positioning of an example footplate against a surface of the eye for treating various tissues or regions of the eye with laser light.

FIG. 4B provides a schematic front illustration of an eye, showing the pupil and approximate positions of various tissues, bodies, and regions of the eye along with an example footplate positioned on a surface of the eye and showing both radial and tangential motion of the footplate for treating multiple tissues or regions of the eye with laser light.

FIG. 4C provides a schematic illustration of an example footplate, showing a single aperture and approximate position within the footplate.

FIG. 5 provides a schematic illustration of an exemplary optical probe according to embodiments of the present technology.

### DETAILED DESCRIPTION OF THE INVENTION

During normal eye function, aqueous humor, a fluid, is continuously generated inside the eye by the ciliary body. Aqueous humor fills the front chambers of the eyeball and travels from the posterior chamber, where it is generated, through the pupil to the anterior chamber between the cornea and the iris. The fluid exits through the trabecular meshwork, a spongy tissue located at the base of the cornea and adjacent to the iris. When aqueous humor builds up in the eye, the intraocular pressure increases, which can result in damage to the optic nerve, a condition generally referred to as glaucoma. Glaucoma can develop when the rate at which aqueous humor is generated by the ciliary body is greater than the rate at which aqueous humor exits through the trabecular meshwork; typically, glaucoma can develop when outflow through the trabecular meshwork is reduced, such as due to degeneration and/or obstruction of the trabecular meshwork. Conventional therapies using phototherapeutic laser treatment (e.g., Selective Laser Trabeculoplasty or Argon Laser Trabeculoplasty) apply laser light to the trabecular meshwork to increase aqueous outflow. In some examples, laser therapies described herein include application of laser light to a different region of the eye, the pars plana. The pars plana is a region of the eye at and beyond the edge of the pars plicata. In some examples, the techniques described herein include application of laser light to the pars plana sequentially or simultaneous with application to other regions.

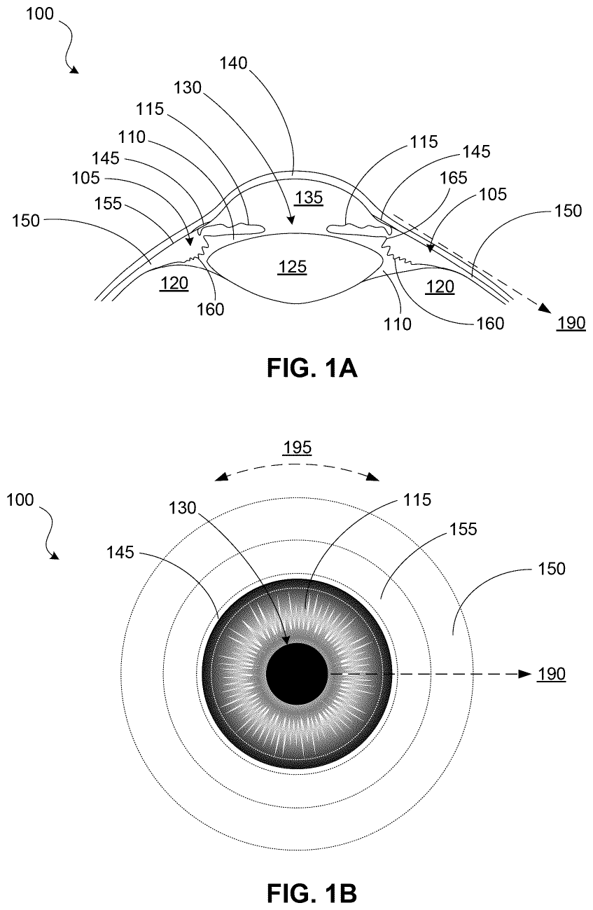

FIG. 1A provides a schematic cross-sectional illustration and FIG. 1B provides a schematic front view illustration showing approximate positions of various portions of the eye 100, including the ciliary body 105, the posterior chamber 110, the iris 115, the vitreous chamber 120, the lens 125, the pupil 130, the anterior chamber 135, the cornea 140, the trabecular meshwork 145, and the pars plana 150. The ciliary body 105 may comprise or include other named components, such as the pars plicata 155, positioned between the pars plana 150 and the iris 115, and the and the ciliary processes 160, positioned at the posterior chamber 110 and responsible for generating the aqueous humor. In some definitions, the pars plana 150 is considered a component of the ciliary body 155. The ciliary sulcus 165 is also identified in FIG. 1A and corresponds to a small space between the posterior surface of the base of the iris 115 and the anterior surface of the ciliary body 155.

The technology described herein include application of laser light to at least the pars plana 150 and optionally other regions, such as the trabecular meshwork 145, pars plicata 155, and/or ciliary sulcus, such as to reduce intraocular pressure (IOP), for treatment of glaucoma, to improve outflow of aqueous humor from the anterior chamber 135, and/or reduce buildup of aqueous humor in the posterior chamber 110 and/or anterior chamber 135.

FIG. 1A and FIG. 1B also illustrate one example of a radial direction 190, corresponding to a direction along the surface of eye 100 toward or away from the pupil 130 at a particular lateral position, and FIG. 1B shows an example of a tangential or circumferential direction 195, corresponding to a direction along the surface of eye 100 about or around the pupil 130, iris 115, and/or cornea 140 at a particular radial position.

It will be appreciated that the accompanying figures are not to scale and are provided as two-dimensional schematic illustrations of an eye (a three-dimensional structure) to show approximate positions of various portions of the eye for purposes of explaining various aspects herein. The skilled artisan will know and understand the positions of the various bodies and elements of the eye based on the illustrations despite any positional or illustrated inaccuracies.

Without wishing to be bound by any theory, exposure of laser light to the trabecular meshwork, the ciliary body, the pars plicata, the pars plana, and/or the ciliary sulcus can improve outflow and reduce intraocular pressure, such as by increasing trabecular outflow, increasing uveoscleral outflow, and/or suppressing generation of aqueous humor.

For exposing the various regions of the eye to laser light, an optical probe can be used. The optical probe may comprise, for example, a footplate, one or more optical waveguides, and a laser system. The footplate may include one or more apertures, which can be respectively coupled to the one or more optical waveguides, such as where an optical waveguide is positioned to fit or pass into and/or through the aperture, or adjoin or abut the aperture. The laser system can be coupled to the optical waveguides such as to emit laser light into the optical waveguides so that the laser light can be transmitted through the apertures and onto the eye of a subject. Additional details of use of paralimbal laser probes for treatment of glaucoma are disclosed in PCT International Application Publication No. WO 2020/040690, which is hereby incorporated by reference.

The footplate can be adapted, shaped, or otherwise configured to couple or mate with a surface of the eye. For example, the footplate can have surface contours or curvature that match or approximately match typical eye contours or curvature, such that a base of the footplate can rest on the mucous membrane or sclera of the eye. The footplate can also have edge contours or curvature that match the contours or curvature of the corneal limbus, which can allow for directly positioning the footplate adjacent to the corneal limbus. In some example, various edges or corners of the footplate can be rounded to avoid application of undesirable amounts of pressure or force to the eye as the footplate is moved radially or tangentially across the eye surface and limit tissue damage due to interaction with an edge or corner of the footplate. In this way, a physician can position the footplate on the eye of a patient to expose various eye tissues or regions to the laser light and/or move the footplate across the surface of the eye (e.g., along a tangential and/or circumferential direction) for treatment of the full circumference or radial thickness of a tissue or region of the eye. The footplate can comprise any suitable material, such as a metal, a ceramic, a plastic, a polymer, or the like. In examples, the footplate can comprise an opaque material or a transparent or partially transparent material. In embodiments, the footplate may be removable (e.g. releasably attached) to the optical probe, such that the footplate may be removed and/or replaced. Such an orientation may allow for the footplate to be considered to be “disposable” and allow for replacing the footplate between treatments or patients.

The footplate can have any suitable dimensions, though particular or typical dimensions may be incorporated into the footplate in order to position the apertures at desirable locations for treatment of particular tissues or regions of the eye with laser light. For example, a length and/or width of the footplate and position of the apertures within the footplate can be adapted to match a position of certain tissues or regions, such as a trabecular meshwork, a pars plicata, a pars plana, a ciliary body, and/or a ciliary sulcus with the apertures, such as when the footplate is positioned against or adjacent to the corneal limbus.

Similarly, an angle of the aperture through the footplate can be adapted to allow for laser light to be directed onto certain tissues or regions by virtue of the angle at which the laser light passes through the aperture. In this way, laser light can be directed towards certain tissues or regions that do not fall directly below the footplate. In some examples, the apertures can be positioned within the footplate such that the various regions or tissues of the eye can be treated as the footplate is moved across the eye, such as in a tangential or circumferential direction about the pupil, iris, cornea, etc., to allow for treatment of tissues circumferentially, such as in multiple or all quadrants of the eye.

Any suitable waveguides can be used with the optical probes described herein. Example waveguides may include optical fibers of any configuration and comprising any suitable material for guiding laser light from a laser system and an emitting the laser light onto an eye. Example optical fibers include single-mode and multi-mode optical fibers, such as comprising core-cladded structures, photonic crystal structures, or the like. Example optical fibers may comprise suitable materials, such as glass, silica, plastic or the like.

In some examples, the incident power of the laser light emitted onto the eye, the output power of the laser light, and/or the power consumption by the laser system may be precisely controlled. In some examples, feedback mechanisms are included in the optical probe to ensure a stable treatment power level during treatment, such as where a power consumption by the laser system, an intensity of the laser light, or the like are monitored to allow for precise control of the treatment power.

Any suitable laser system may be used with the optical probes described herein. In examples, the output wavelength of the laser system is from 700 nm to 900 nm, such as from 700 nm to 705 nm, from 705 nm to 710 nm, from 710 nm to 715 nm, from 715 nm to 720 nm, from 720 nm to 725 nm, from 725 nm to 730 nm, from 730 nm to 735 nm, from 735 nm to 740 nm, from 740 nm to 745 nm, from 745 nm to 750 nm, from 750 nm to 755 nm, from 755 nm to 760 nm, from 760 nm to 765 nm, from 765 nm to 770 nm, from 770 nm to 775 nm, from 775 nm to 780 nm, from 780 nm to 785 nm, from 785 nm to 790 nm, from 790 nm to 795 nm, from 795 nm to 800 nm, from 800 nm to 805 nm, from 805 nm to 810 nm, from 810 nm to 815 nm, from 815 nm to 820 nm, from 820 nm to 825 nm, from 825 nm to 830 nm, from 830 nm to 835 nm, from 835 nm to 840 nm, from 840 nm to 845 nm, from 845 nm to 850 nm, from 850 nm to 855 nm, from 855 nm to 860 nm, from 860 nm to 865 nm, from 865 nm to 870 nm, from 870 nm to 875 nm, from 875 nm to 880 nm, from 880 nm to 885 nm, from 885 nm to 890 nm, from 890 nm to 895 nm, from 895 nm to 900 nm, or any ranges or values therebetween. In some examples, the laser system may be controlled or factory calibrated to deliver a specific amount of wattage or energy of laser light on the contact surface consistently. Pulsed or continuous wave lasers may be used, depending on the desired configuration. Example output powers may include, but are not limited to 1.5 W to 3.0 W, such as from 1.5 W to 1.75 W, from 1.75 W to 2.0 W, from 2.0 W to 2.25 W, from 2.25 W to 2.5 W, from 2.5 W to 2.75 W, or from 2.75 W to 3.0 W, or any ranges or values therebetween, and may be at a duty cycle of from 25% to 45%, such as from 25% to 30%, from 30% to 35%, from 35% to 40%, from 40% to 45%, or any ranges or values therebetween.

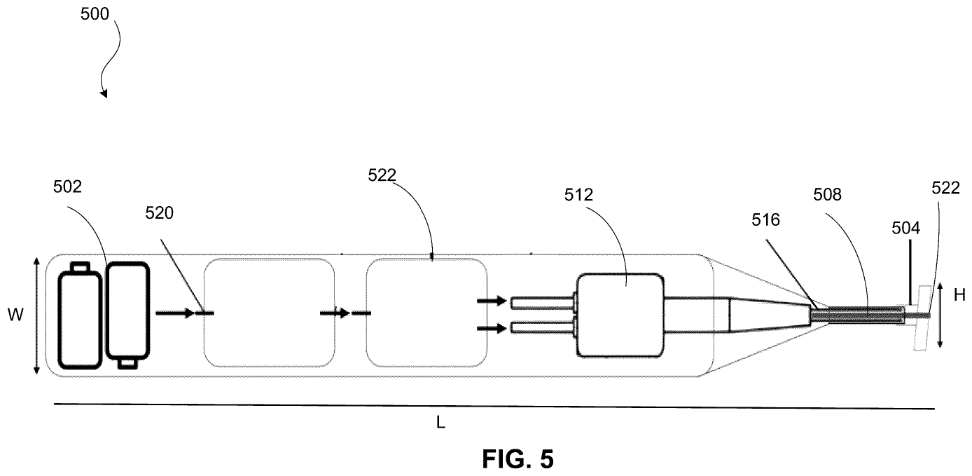

In some examples, a control system, such as control system 520 of FIG. 5, is incorporated into the optical probe to control an output power of laser light emitted by the optical probe and/or onto the eye. The control system can be used to modulate or maintain a fixed output power, such as to ensure treatment using the optical probe occurs at a desired output power. In some examples, controlling the output power may include limiting output of laser light, such as to prevent laser emission, as may be illustrated by device limiter 522 of FIG. 5. Such a configuration may be useful when the laser system's output power level is uncontrollable, unstable, or falls below a threshold or target power level. For example, some laser systems may exhibit a power loss based on duration of use, such that the output power naturally decreases over time. In some examples, increasing a power provided to the laser system may allow, at least in part, for maintaining an output level of the laser light at a desirable level, but over time and based on a usage time, for example, of the laser system, the output power level may fall below a target or useful level, such as a level below which exposure of the eye to laser light is ineffective for treating high intraocular pressure according to aspects described above. In such embodiments, the device limiter may prevent usage of the optical probe after output drops below a control level, preventing usage of the optical probe when the target power output cannot be achieved. For instance, the device limiter 522 may monitor the amount of power left if a power source having a power bank is utilized (discussed in greater detail in FIG. 5), the time the laser light has been utilized or triggered, as well as the amount of output power utilized.

Namely, in cases where an output power level of a laser system falls below a target or useful level, it may be desirable to prevent the laser from outputting any laser light at all, effectively limiting the lifespan of the probe. Such a configuration may arise based on a number of treatment applications, and thus some examples may limit the laser to outputting any light only for a fixed or target amount of time or a fixed or target number of treatments, beyond which the control system will control the output such that no laser light is emitted by the laser system. Embodiments such as these may be useful for maintaining the optical probe in a high-performance configuration where treatments are effective and prevent ineffective or deleterious treatments using optical probes where the output power has fallen below a suitable or target level. In examples, a control system may allow operation of a probe for a fixed number of treatments, such as 3 treatments or 5 treatments, after which the control system will lock out any laser output from the device.

Several example non-limiting embodiments of various optical probes are described. Although such optical probes may be described as separate embodiments, it will be appreciated that various features and aspects of the different embodiments can be combined by those of skill in the art according to the disclosed features and aspects herein.

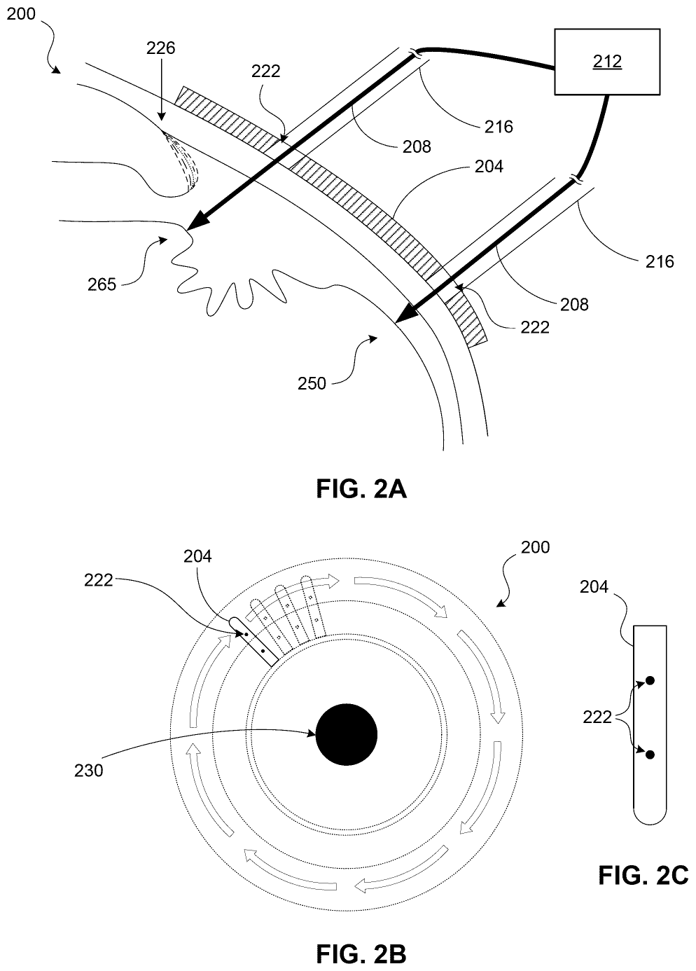

FIG. 2A, FIG. 2B, and FIG. 2C provide details of various components of a first example optical probe for treatment of an eye with laser light. In some examples, the first example laser probe may provide dual lasers that are spaced apart such that the lasers target the pars plana and a second component of the eye concurrently (e.g., the trabecular meshwork, the pars plicata, the ciliary sulcus, etc.). The laser and footplate can be moved parallel to the pars plana in each quadrant of the eye during the surgical process, for example where the optical probe is moved in a circumferential direction about the pupil, iris, etc.

FIG. 2A provides a partial schematic cross-sectional illustration of an anterior portion of an eye 200, showing positioning of an example footplate 204 of the first optical probe against a surface of the eye 200 for treating different tissues or regions of the eye 200 with laser light 208 emitted by laser system 212 into waveguides 216. Waveguides 216 are coupled to footplate 204 such that the laser light 208 is emitted through apertures 222 and onto tissues of eye 200. Waveguides 216 may be coupled to footplate 204 orthogonally or at an angle. In some examples, positioning waveguides 216 at an angle to footplate 204 may be useful for directing laser light 208 onto particular structures, regions, or tissues of the eye. As depicted, footplate 204 is positioned adjacent to a surface of the eye 200 and against or adjacent to the corneal limbus 226, with the waveguides 216 positioned at an angle of about 70° from footplate 204, such as to direct laser light 208 onto pars plana 250 or ciliary sulcus 265. It will be appreciated an angle of 70° is merely one non-limiting example and that any suitable angle (e.g., from 1° to) 90° and position of the apertures 222 can be implemented to direct the laser light onto desirable tissues or regions.

FIG. 2B provides a schematic front illustration of the eye 200. FIG. 2B shows only pupil 230, so as not to obscure other details, and shows approximate border positions for various tissues, bodies, and regions of the eye, such as the trabecular meshwork, pars plana, and ciliary body, similar to the depiction in FIG. 1B. FIG. 2B also shows the example footplate 204 of the first example optical probe positioned on the surface of the eye and schematically showing circumferential motion of the footplate 204 for treating different regions or quadrants of the eye 200 with laser light. Footplate 204 is depicted in a radial orientation for simultaneously treating multiple different tissues, bodies, or regions of the eye with laser light at the same time.

FIG. 2C provides a schematic illustration of an example footplate 204, showing approximate positions of apertures 222 within the footplate 204 according to some embodiments. Without limitation, footplate 204 can have any suitable dimensions, such as a width of about 1 mm to about 3 mm, such as from 1.0 mm to 1.2 mm, from 1.2 mm to 1.4 mm, from 1.4 mm to 1.6 mm, from 1.6 mm to 1.8 mm, from 1.8 mm to 2.0 mm, from 2.0 mm to 2.2 mm, from 2.2 mm to 2.4 mm, from 2.4 mm to 2.6 mm, from 2.6 mm to 2.8 mm, or from 2.8 mm to 3.0 mm, and a length of about 5.5 mm to about 7.5 mm, such as from 5.5 mm to 5.7 mm, from 5.7 mm to 5.9 mm, from 5.9 mm to 6.1 mm, from 6.1 mm to 6.3 mm, from 6.3 mm to 6.5 mm, from 6.5 mm to 6.7 mm, from 6.7 mm to 6.9 mm, from 6.9 mm to 7.1 mm, from 7.1 mm to 7.3 mm, or from 7.3 mm to 7.5 mm, or any ranges or values therebetween. Apertures 222 are depicted as positioned on a center line of footplate 204, but can be positioned at any other or suitable position. Without limitation, one aperture 222 of footplate 204 can be positioned about 1 mm to about 2 mm from an edge of footplate 204, such as from 1.0 mm to 1.1 mm, from 1.1 mm to 1.2 mm, from 1.2 mm, to 1.3 mm, from 1.3 mm to 1.4 mm, from 1.4 mm to 1.5 mm, from 1.5 mm to 1.6 mm, from 1.6 mm to 1.7 mm, from 1.7 mm to 1.8 mm, from 1.8 mm to 1.9 mm, or from 1.9 mm to 2.0 mm, and the other aperture 222 of footplate 204 can be positioned about 3 mm to about 3.5 mm from an edge of footplate 204, such as from 3 mm to 3.1 mm, from 3.1 mm to 3.2 mm, from 3.2 mm to 3.3 mm, from 3.3 mm to 3.4 mm, or from 3.4 mm to 3.5 mm. Optionally, a distance between apertures is about 1.5 mm to about 2.5 mm, such as from 1.5 mm to 1.6 mm, from 1.6 mm to 1.7 mm, from 1.7 mm to 1.8 mm, from 1.8 mm to 1.9 mm, from 1.9 mm to 2.0 mm, from 2.0 mm to 2.1 mm, from 2.1 mm to 2.2 mm, from 2.2 mm, to 2.3 mm, from 2.3 mm to 2.4 mm, or from 2.4 mm to 2.5 mm, or any ranges or values therebetween.

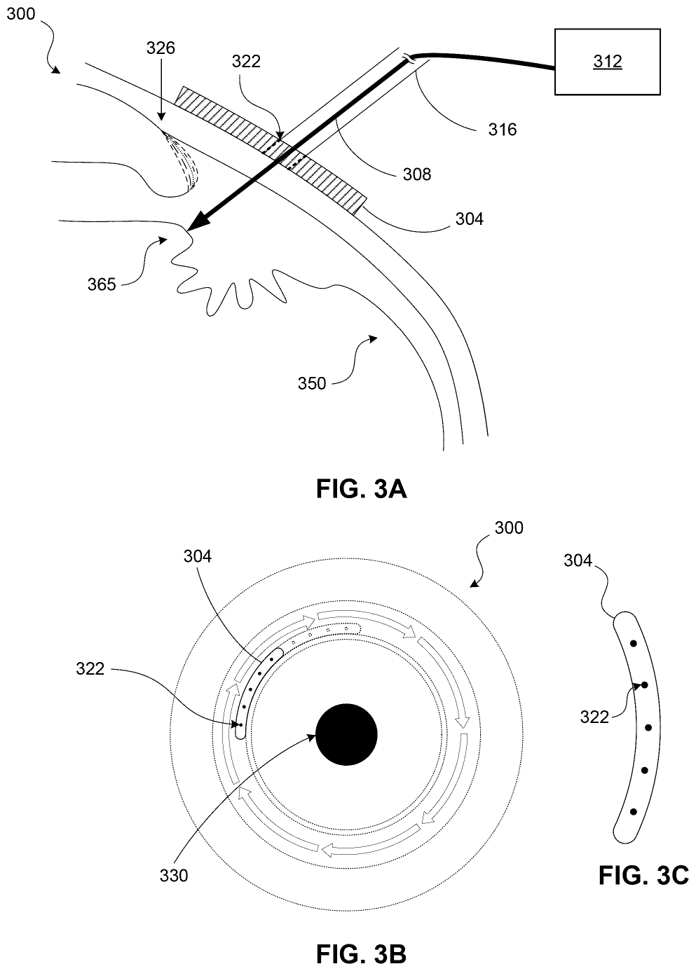

FIG. 3A, FIG. 3B, and FIG. 3C provide details of various components of a second example optical probe for treatment of an eye with laser light. In some examples, the second example laser probe may provide a plurality of lasers that are spaced apart such that the lasers target different circumferential positions of a single tissue region concurrently (e.g., the pars plana, the trabecular meshwork, the pars plicata, the ciliary sulcus, etc.), such as part of one quadrant of the eye or a full amount of one quadrant of the eye. The laser and footplate can be moved tangentially or circumferentially in each quadrant of the eye during the surgical process, for example where the optical probe is moved in a circumferential direction about the pupil, iris, etc. to expose each quadrant to laser light. The laser and footplate can also be moved radially (e.g., inward or outward relative to the pupil) to sequentially expose different tissues to laser light and the circumferential motion at each radial position can be repeated in each quadrant of the eye during the surgical process.

FIG. 3A provides a partial schematic cross-sectional illustration of an anterior portion of an eye 300, showing positioning of an example footplate 304 of the second optical probe against a surface of the eye 300 for treating tissues or regions of a partial quadrant of the eye 300 with laser light 308 emitted by laser system 312 into waveguides 316. Waveguides 316 are coupled to footplate 304 such that the laser light 308 is emitted through apertures 322 and onto tissues of eye 300. Waveguides 316 may be coupled to footplate 304 orthogonally or at an angle. Again, positioning waveguides 316 at an angle to footplate 304 may be useful for directing laser light 308 onto particular structures, regions, or tissues of the eye. As depicted, footplate 304 is positioned adjacent to a surface of the eye 300 and against or adjacent to the corneal limbus 326, with the waveguides 316 positioned at an angle of about 70° from footplate 304, such as to direct laser light 308 onto ciliary sulcus 365. It will be appreciated an angle of 70° is merely one non-limiting example and that any suitable angle (e.g., from 1° to) 90° and position of the apertures 322 can be implemented to direct the laser light onto desirable tissues or regions.

FIG. 3B provides a schematic front illustration of the eye 300. FIG. 3B shows only pupil 330, so as not to obscure other details, and shows approximate border positions for various tissues, bodies, and regions of the eye, such as the trabecular meshwork, pars plana, and ciliary body, similar to the depiction in FIG. 1B. FIG. 3B also shows the example footplate 304 of the second example optical probe positioned on the surface of the eye and schematically showing circumferential motion of the footplate 304 for treating different regions or quadrants of the eye 300 with laser light. Footplate 304 is depicted in a circumferential orientation for simultaneously treating multiple positions of the same tissue, body, or region of the eye with laser light at the same time.

FIG. 3C provides a schematic illustration of an example footplate 304, showing approximate positions of apertures 322 within the footplate 304 according to some embodiments. Without limitation, footplate 304 can have any suitable dimensions and/or number of apertures. As depicted, footplate 304 include 5 apertures and has a width of about 1 mm to about 3 mm, such as from 1.0 mm to 1.2 mm, from 1.2 mm to 1.4 mm, from 1.4 mm, to 1.6 mm, from 1.6 mm to 1.8 mm, from 1.8 mm to 2.0 mm, from 2.0 mm to 2.2 mm, from 2.2 mm to 2.4 mm, from 2.4 mm, to 2.6 mm, from 2.6 mm to 2.8 mm, or from 2.8 mm to 3.0 mm, and a length of about 4 mm to about 10 mm, such as from 4.0 mm to 4.5 mm, from 4.5 mm to 5.0 mm, from 5.0 mm to 5.5 mm, from 5.5 mm to 6.0 mm, from 6.0 mm to 6.5 mm, from 6.5 mm to 7.0 mm, from 7.0 mm to 7.5 mm, from 7.5 mm to 8.0 mm, from 8.0 mm to 8.5 mm, from 8.5 mm to 9.0 mm, from 9.0 mm to 9.5 mm, or from 9.5 mm to 10.0 mm, or any ranges or values therebetween, albeit footplate 304 has a curved geometry suitable for matching an approximate circumference of the iris, or slightly larger. Apertures 322 are depicted as positioned on a center line of footplate 304, but can be positioned at any other or suitable position. Without limitation, apertures 322 of footplate 304 can be positioned about 1 mm to about 3 mm from an edge of footplate 304, such as from 1.0 mm to 1.2 mm, from 1.2 mm to 1.4 mm, from 1.4 mm, to 1.6 mm, from 1.6 mm to 1.8 mm, from 1.8 mm to 2.0 mm, from 2.0 mm to 2.2 mm, from 2.2 mm to 2.4 mm, from 2.4 mm, to 2.6 mm, from 2.6 mm to 2.8 mm, or from 2.8 mm to 3.0 mm, and apertures 322 can be positioned about 1 mm to about 3 mm from one another, such as from 1.0 mm to 1.2 mm, from 1.2 mm to 1.4 mm, from 1.4 mm, to 1.6 mm, from 1.6 mm to 1.8 mm, from 1.8 mm to 2.0 mm, from 2.0 mm to 2.2 mm, from 2.2 mm to 2.4 mm, from 2.4 mm, to 2.6 mm, from 2.6 mm to 2.8 mm, or from 2.8 mm to 3.0 mm, or any ranges or values therebetween.

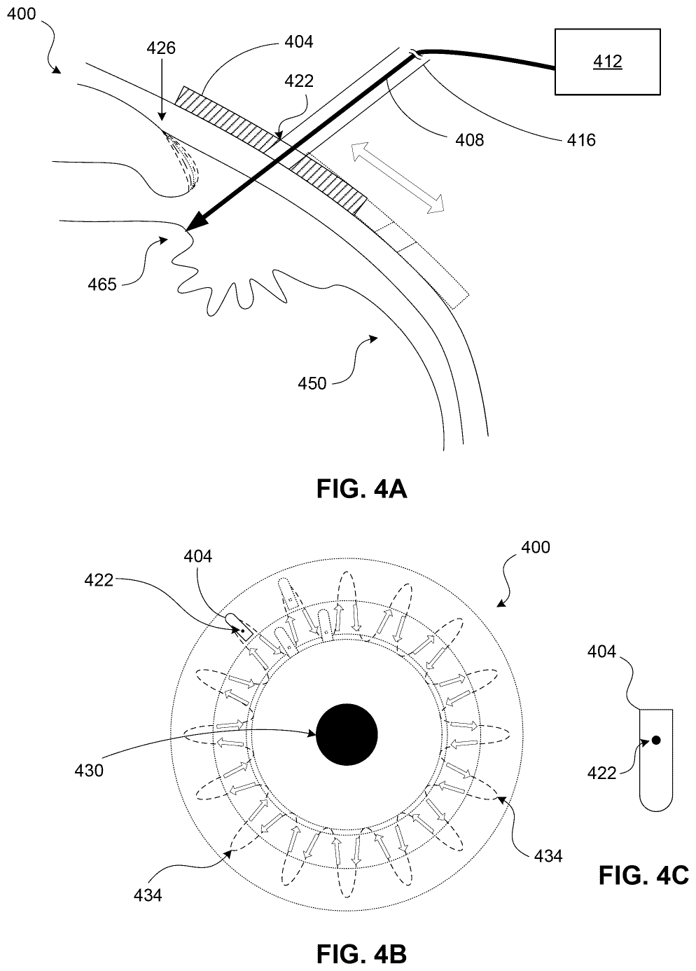

FIG. 4A, FIG. 4B, and FIG. 4C provide details of various components of a third example optical probe for treatment of an eye with laser light. In some examples, the third example laser probe may provide one laser to target a single tissue region at one time (e.g., the pars plana, the trabecular meshwork, the pars plicata, the ciliary sulcus, etc.). The laser and footplate are depicted as moving both radially as well as tangentially or circumferentially to expose multiple tissues, bodies, or regions of the eye to laser light in sequence, such as by moving along a zig-zag or other complex path across and about the eye.

FIG. 4A provides a partial schematic cross-sectional illustration of an anterior portion of an eye 400, showing positioning of an example footplate 404 of the third optical probe against a surface of the eye 400 for treating tissues or regions of the eye 400 with laser light 408 emitted by laser system 412 into a waveguides 416. Waveguide 416 is coupled to footplate 404 such that the laser light 408 is emitted through the aperture 422 and onto the eye 400. Waveguide 416 may be coupled to footplate 404 orthogonally or at an angle. Again, positioning waveguide 416 at an angle to footplate 404 may be useful for directing laser light 408 onto particular structures, regions, or tissues of the eye. As depicted, footplate 404 is positioned adjacent to a surface of the eye 400 and against or adjacent to the corneal limbus 426, with the waveguide 416 positioned at an angle of about 70° from footplate 404, such as to direct laser light 408 onto ciliary sulcus 465. It will be appreciated an angle of 70° is merely one non-limiting example and that any suitable angle (e.g., from 1° to) 90° and position of the aperture 422 can be implemented to direct the laser light onto desirable tissues or regions.

FIG. 4B provides a schematic front illustration of the eye 400. FIG. 4B shows only pupil 430, so as not to obscure other details, and shows approximate border positions for various tissues, bodies, and regions of the eye, such as the trabecular meshwork, pars plana, and ciliary body, similar to the depiction in FIG. 1B. FIG. 4B also shows the example footplate 404 of the third example optical probe positioned on the surface of the eye 400 and schematically showing combined radial and circumferential motion of the footplate 404 along a zig-zag path 434 for treating different regions and/or quadrants of the eye 400 with laser light. Footplate 404 is depicted in a radial orientation for treating a single tissue, body, or region of the eye 400 with laser light, but motion along zig-zag path 434 can provide for full treatment of a variety of tissues, bodies, or regions of the eye 400 in sequence.

FIG. 4C provides a schematic illustration of an example footplate 404, showing the approximate position of aperture 422 within the footplate 404 according to some embodiments. Without limitation, footplate 404 can have any suitable dimensions and/or number of apertures. As depicted, footplate 404 include a single aperture and has a width of about 1 mm to about 3 mm, such as from 1.0 mm to 1.2 mm, from 1.2 mm to 1.4 mm, from 1.4 mm, to 1.6 mm, from 1.6 mm to 1.8 mm, from 1.8 mm to 2.0 mm, from 2.0 mm to 2.2 mm, from 2.2 mm to 2.4 mm, from 2.4 mm, to 2.6 mm, from 2.6 mm to 2.8 mm, or from 2.8 mm to 3.0 mm, and a length of about 2 mm to about 4 mm, such as from 2.0 mm to 2.2 mm, from 2.2 mm to 2.4 mm, from 2.4 mm, to 2.6 mm, from 2.6 mm to 2.8 mm, from 2.8 mm to 3.0 mm, from 3.0 mm to 3.2 mm, from 3.2 mm to 3.4 mm, from 3.4 mm, to 3.6 mm, from 3.6 mm to 3.8 mm, or from 3.8 mm to 4.0 mm or any ranges or values therebetween. Aperture 322 is depicted as positioned on a center line of footplate 404, but can be positioned at any other or suitable position. Without limitation, aperture 422 of footplate 404 can be positioned centrally within footplate 404 or about 1 mm to about 3 mm from the edges of footplate 204, such as from 1.0 mm to 1.2 mm, from 1.2 mm to 1.4 mm, from 1.4 mm, to 1.6 mm, from 1.6 mm to 1.8 mm, from 1.8 mm to 2.0 mm, from 2.0 mm to 2.2 mm, from 2.2 mm to 2.4 mm, from 2.4 mm, to 2.6 mm, from 2.6 mm to 2.8 mm, or from 2.8 mm to 3.0 mm, or any ranges or values therebetween.

FIG. 5 provides details of various components of an exemplary optical probe 500 for treatment of an eye with laser light. In the illustrated embodiment, which may be applicable with any one or more of the probes and footplates discussed as well, the optical probe 500 may considered to be “hand-held” due to the compact size, as compared to a stationary device. Such an orientation may allow for greater accessibility in treatment as the device may be easily transported, and may also improve the comfort and positioning of the optical probe.

In embodiments, the optical probe 500 may have a length L of about 10 cm to about 30 cm, such as greater than or about 10 cm, greater than or about 11 cm, greater than or about 12 cm, greater than or about 13 cm, greater than or about 14 cm, greater than or about 15 cm, greater than or about 16 cm, greater than or about 17 cm, greater than or about 18 cm, greater than or about 19 cm, greater than or about 20 cm, greater than or about 21 cm, greater than or about 22 cm, greater than or about 23 cm, greater than or about 24 cm, greater than or about 25 cm, or such as about 30 cm or less, such as less than or about 29 cm, less than or about 28 cm, less than or about 27 cm, less than or about 26 cm, less than or about 26 cm, less than or about 25 cm, less than or about 24 cm, less than or about 23 cm, less than or about 22 cm, less than or about 21 cm, less than or about 20 cm, or any ranges or values therebetween.

Furthermore, in embodiments, the optical probe 500 may have a width W of the body portion of about 1 cm to about 10 cm, such as greater than or about 1 cm, greater than or about 2 cm, greater than or about 3 cm, greater than or about 4 cm, greater than or about 5 cm, or such as less than or about 10 cm, less than or about 9 cm, less than or about 8 cm, less than or about 7 cm, less than or about 6 cm, less than or about 5 cm, less than or about 4 cm, less than or about 3 cm, or any ranges or values therebetween.

In addition, in embodiments, the optical probe 500 may have a footplate 504 having a height H of about 0.1 cm to about 1 cm, such as greater than or about 0.1 cm, greater than or about 0.2 cm, greater than or about 0.3 cm, greater than or about 0.4 cm, greater than or about 0.5 cm, or such as less than or about 1 cm, less than or about 0.9 cm, less than or about 0.8 cm, less than or about 0.7 cm, less than or about 0.6 cm, less than or about 0.5 cm, or any ranges or values therebetween. Nonetheless, in embodiments, the footplate 504 may have any one or more of the footplate dimensions discussed herein.

In some examples, the optical probe 500 may provide a plurality of lasers 508 that are spaced apart such that the lasers target different circumferential positions of a single tissue region concurrently (e.g., the pars plana, the trabecular meshwork, the pars plicata, the ciliary sulcus, etc.), such as part of one quadrant of the eye or a full amount of one quadrant of the eye, or may only have a single laser light 508 with a single aperture 522, as discussed above. The laser and footplate can be moved tangentially or circumferentially in each quadrant of the eye during the surgical process, for example where the optical probe 500 is moved in a circumferential direction about the pupil, iris, etc. to expose each quadrant to laser light. The laser and footplate can also be moved radially (e.g., inward or outward relative to the pupil) to sequentially expose different tissues to laser light and the circumferential motion at each radial position can be repeated in each quadrant of the eye during the surgical process.

FIG. 5 provides a partial schematic cross-sectional illustration of an optical probe 500 for treating tissues or regions of a partial quadrant of the eye with laser light 508 emitted by laser system 512 into waveguides 516. Waveguides 516 are coupled to footplate 504 such that the laser light 508 is emitted through apertures 522 and onto tissues of eye. Waveguides 516 may be coupled to footplate 504 orthogonally or at an angle. Again, positioning waveguides 5516 at an angle to footplate 504 may be useful for directing laser light 508 onto particular structures, regions, or tissues of the eye. As discussed above, footplate 504 may be positioned adjacent to a surface of the eye and against or adjacent to the corneal limbus thereof, with the waveguides 516 positioned at an angle so as to direct laser light 508 onto a desired region of the eye, which may include any one or more of the eye structures discussed above.

As illustrated, in embodiments, a handheld device, such as the illustration of FIG. 5, may be portable, and may therefore contain a portable power storage device 502. The power storage device 502 may include one or more batteries or other power banks. However, in embodiments, the optical device 500 may include one or more power cords that allow for the device to remain portable, while also removing the need for a power storage device. Nonetheless, the power storage device 502 may further improve the accessibility of the device, and may be utilized as the device limiter may carefully track and control the power and output of the device.

Again, it will be appreciated that the first, second, third, and fourth optical probes described above are merely examples and are not intended to limit the present disclosure, In some examples, different features of the first, second, third, and fourth optical probes can be combined according to any suitable combination, such as where multiple apertures are included in a footplate that is moved along a zig-zag or other complex path, or where multiple radial rows of apertures are included in a curved footplate that is moved circumferentially about the eye, or where any one or more of the designs are desired to be portable.

In the preceding description, for the purposes of explanation, numerous details have been set forth in order to provide an understanding of various embodiments of the present technology. It will be apparent to one skilled in the art, however, that certain embodiments may be practiced without some of these details, or with additional details.

Having disclosed several embodiments and examples, it will be recognized by those of skill in the art that various modifications, alternative constructions, and equivalents may be used without departing from the spirit of the embodiments. Additionally, a number of well-known processes and elements have not been described in order to avoid unnecessarily obscuring the present technology. Accordingly, the above description should not be taken as limiting the scope of the technology.

Where a range of values is provided, it is understood that each intervening value, to the smallest fraction of the unit of the lower limit, unless the context clearly dictates otherwise, between the upper and lower limits of that range is also specifically disclosed. Any narrower range between any stated values or unstated intervening values in a stated range and any other stated or intervening value in that stated range is encompassed. The upper and lower limits of those smaller ranges may independently be included or excluded in the range, and each range where either, neither, or both limits are included in the smaller ranges is also encompassed within the technology, subject to any specifically excluded limit in the stated range. Where the stated range includes one or both of the limits, ranges excluding either or both of those included limits are also included.

As used herein and in the appended claims, the singular forms “a”, “an”, and “the” include plural references unless the context clearly dictates otherwise. Thus, for example, reference to “a material” includes a plurality of such materials, and reference to “the nutrient” includes reference to one or more nutrients and equivalents thereof known to those skilled in the art, and so forth.

Also, the words “comprise(s)”, “comprising”, “contain(s)”, “containing”, “include(s)”, and “including”, when used in this specification and in the following claims, are intended to specify the presence of stated features, integers, components, or operations, but they do not preclude the presence or addition of one or more other features, integers, components, operations, acts, or groups.

What is claimed is:

1. A system comprising:
a footplate, the footplate shaped to mate with a surface of an eye, the footplate including one or more apertures;
one or more optical waveguides coupled to the footplate and positioned to transmit laser light through the one or more apertures and expose at least a pars plana of the eye to laser light when the footplate is mated with the surface of the eye; and
a laser system coupled to the one or more optical waveguides to emit laser light into the one or more optical waveguides for transmission to the eye through the one or more apertures.

2. The system of claim 1, wherein the footplate is shaped to mate with a sclera of the eye at or adjacent to a corneal limbus of the eye.

3. The system of claim 1, wherein one or more dimensions of the footplate and/or a position of one or more apertures within the footplate are configured to position at least one aperture of the footplate above the pars plana when the footplate is mated to the surface of the eye.

4. The system of claim 1, wherein one or more dimensions of the footplate and/or a position of the one or more apertures within the footplate are configured to position at least one aperture of the footplate above the pars plana and at least one aperture of the footplate above a pars plicata of the eye when the footplate is mated to the surface of the eye.

5. The system of claim 1, wherein one or more dimensions of the footplate and/or a position of the one or more apertures within the footplate are configured to position at least one aperture of the footplate above the pars plana and at least one aperture of the footplate above a trabecular meshwork of the eye when the footplate is mated to the surface of the eye.

6. The system of claim 1, wherein one or more dimensions of the footplate and/or a position of the one or more apertures within the footplate are configured to position at least one aperture of the footplate above a pars plicata of the eye and at least one aperture of the footplate above a trabecular meshwork of the eye when the footplate is mated to the surface of the eye.

7. The system of claim 1, wherein one or more dimensions of the footplate and/or a position of the one or more apertures within the footplate are configured to position a plurality of apertures of the footplate above the pars plana or above a pars plicata of the eye when the footplate is mated to the surface of the eye.

8. The system of claim 1, wherein the footplate is adapted to move tangentially or circumferentially along the surface of the eye, parallel to the pars plana, to sequentially expose at least the pars plana in a plurality of quadrants of the eye to laser light.

9. The system of claim 1, wherein the footplate is adapted to move tangentially or circumferentially along the surface of the eye, parallel to a ciliary sulcus of the eye, to sequentially expose at least the ciliary sulcus in a plurality of quadrants of the eye to laser light.

10. The system of claim 1, wherein tangential or circumferential motion of the footplate corresponds to a translation along the surface of the eye about a pupil of the eye at a fixed radial position.

11. The system of claim 1, wherein the footplate is adapted to move radially along the surface of the eye to expose the pars plana and a pars plicata of the eye to laser light.

12. The system of claim 1, wherein the footplate is adapted to move radially along the surface of the eye to expose the pars plana, a pars plicata of the eye, and a trabecular meshwork of the eye to laser light.

13. The system of claim 1, wherein radial motion of the footplate corresponds to a translation along the surface of the eye toward or away from a pupil of the eye at a fixed lateral position.

14. The system of claim 1, wherein the footplate is adapted to move both radially and tangentially along the surface of the eye to expose the pars plana and a trabecular meshwork of the eye in a plurality of quadrants of the eye to laser light.

15. The system of claim 1, wherein the footplate is adapted to move radially and tangentially along the surface of the eye to expose the pars plana, a pars plicata of the eye, and a trabecular meshwork of the eye to laser light.

16. The system of claim 1, wherein the footplate is adapted to move both radially and tangentially in a zig-zag path along the surface of the eye.

17. The system of claim 1, further comprising a control system configured to control an output power of laser light emitted onto the eye.

18. The system of claim 17, wherein the control system is configured to limit operation of the laser system to a fixed number of treatments to maintain a treatment power of the laser light above a threshold level.

19. A method comprising:
positioning the footplate of the system of claim 1 against the surface of an eye; and
causing actuation of the laser system to expose laser light onto at least the pars plana of the eye through the one or more apertures.

20. The method of claim 19, further comprising:
moving the footplate from a first position against the surface of the eye to a second position against the surface of the eye; and
causing actuation of the laser system to expose laser light onto the eye through the one or more apertures at the second position, wherein exposing the laser light onto the eye through the one or more apertures at the second position includes exposing the pars plana, a trabecular meshwork of the eye, a pars plicata of the eye, and/or a ciliary sulcus of the eye to the laser light.
